(19) United States
(12) Plant Patent Application Publication
(10) Pub. No.: US 2002/0133856 P1
(43) Pub. Date: Sep. 19, 2002

(54) LAVANDULA PLANT NAMED 'WALVERA'

(76) Inventors: Albert Timothy Alan Crowther, Arundel (GB); David Ralph Tristram, Arundel (GB)

Correspondence Address: MARK P. BOURGEOIS, P.O. BOX 95, OSCEOLA, IN, 46561, US

(21) Appl. No.: 09/804,916

(22) Filed: Mar. 13, 2001

(51) Int. Cl. A01H 5/00

(52) U.S. Cl. PLT/226

(57) ABSTRACT

A new cultivar of Lavendula angustifolia plant named ‘Walvera’ that is characterized by broad variegated foliage with gray-green centers and cream-white margins, a mounded, semi-open habit, and compact violet-blue flower spikes. In combination, these traits set it apart from all other existing varieties of Lavendula known to the inventors.

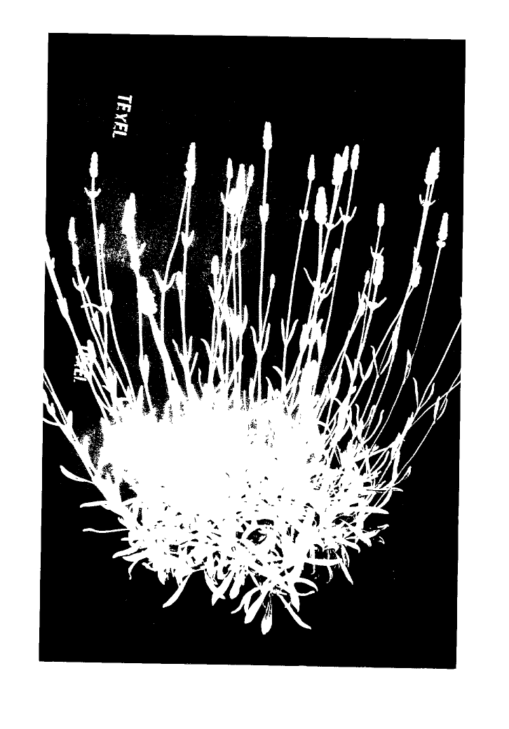

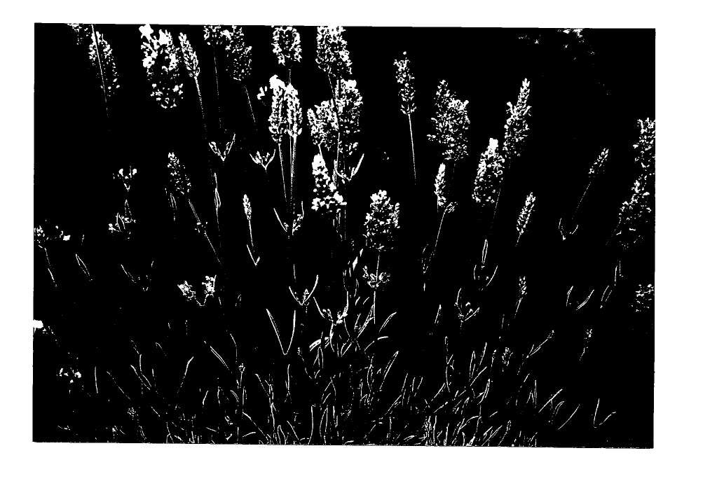

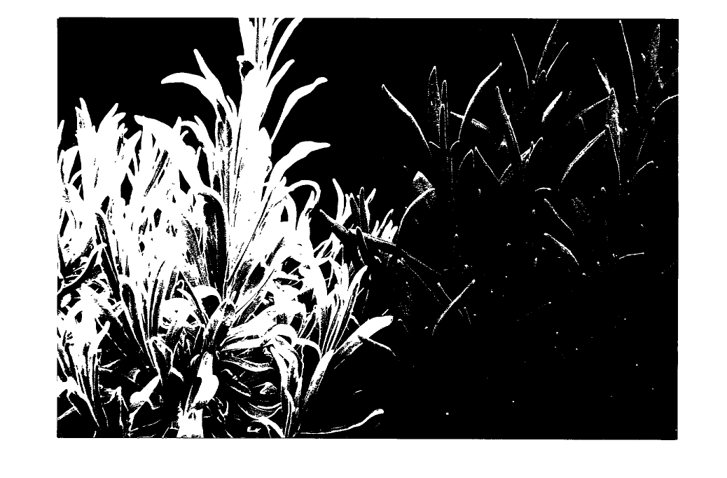

### BACKGROUND OF THE INVENTION

The present invention relates to a new and distinct cultivar of lavender known botanically as Lavendula angustifolia and hereinafter referred to by the cultivar name ‘Walvera’. The new cultivar is a naturally occurring branch sport of Lavendula angustifolia ‘Vera’ (unpatented) and was discovered jointly by the inventors in a cultivated area of West Sussex, United Kingdom in 1993.

The new lavender plant ‘Walvera’ is distinct and unique due to its mounding, semi-open habit, gray-green leaves with cream-white marginal variegation, and violet-blue flowers. The closest comparison is the parent plant Lavendula angustifolia ‘Vera’ (unpatented). ‘Walvera’ is distinguishable from the parent plant, and all other existing varieties known to the inventors, by its variegated foliage.

The new invention was first asexually propagated in West Sussex, United Kingdom by the inventors in 1995. Since that time subsequent generations have been determined fixed and stable.

### SUMMARY OF THE INVENTION

The new lavender ‘Walvera’ is distinguished from all other known varieties of Lavendula, by its characteristic broad leaves with gray-green centers and cream-white variegated margins. This unique trait sets ‘Walvera’ apart from all other existing varieties known to the inventors.

### BRIEF DESCRIPTION OF THE DRAWINGS

The accompanying color photographs illustrate the distinguishing characteristics of the new cultivar ‘Walvera’. The first photograph shows the entire plant from a side view perspective, illustrating the plant habit. The second photograph illustrates the flowers on ‘Walvera’. The third photograph is a comparative photo illustrating the variegated foliage of ‘Walvera’ on the left, and the foliage of the parent plant Lavendula angustifolia ‘Vera’ (unpatented) on the right All photographs are taken using conventional photographic techniques and although foliage colors may appear different from actual colors due to light reflectance, they are as accurate as possible by conventional photography.

### BOTANICAL DESCRIPTION OF THE PLANT

The following is a detailed description of the Lavendula angustifolia cultivar named ‘Walvera’. Data was collected in Arroyo Grande, Calif. from plants grown outdoors in one-gallon containers. Phenotypic differences may be observed with variations in environmental, climatic, and cultural conditions, without however, any difference in genotype. Color determinations are in accordance with The Royal Horticultural Society Color Chart except where general color terms of ordinary dictionary significance are used. The growing requirements are similar to the species and no unusual disease problems have been observed.

Botanical classification: Lavendula angustifolia ‘Walvera’

Common name: Variegated Lavender.

Parentage: A naturally occurring branch sport of Lavendula angustifolia ‘Vera’.

Type: Aromatic perennial shrub.

Use: Ornamental, hedge or border plant.

Growth rate: Vigorous.

Habit: Mounded and semi-open with erect, ascending flower spikes.

Height: 0.3 to 1.0 m in height.

Spread: 0.4 to 0.8 m in width.

Hardiness: Zone 5 to zone 10.

Propagation method: Cuttings.

Root system: Fibrous.

Soil: Plant in well-drained soil.

Light levels: Plant in full sun.

Special needs: None observed.

Time to initiate roots: 14 to 36 days from an unrooted cutting.

Time to develop in a one-gallon container: 6 months from a rooted cutting.

Branching habit: Erect, upright and semi-open.

Seasonal interest: Violet-blue flowers in July and August.

Stem:

Shape.—Square.

Surface.—Puberulent.

Color.—191A.

Size.—2 to 5 mm in diameter.

Foliage:

Arrangement.—Opposite.

Leaf division.—Simple.

Apices.—Acute.

Base.—Attenuate.

Margins.—Entire and mostly revolute.

Leaf shape.—Linear.

Color of mature leaves (upper and lower surfaces).—Center 190A to 191A and margins 11C.

Color of young leaves (lower surfaces).—Center 189B and margins 11D.

Color of young leaves (upper surfaces).—189D.

Upper and lower surfaces.—Puberulent.

Venation.—Prominent mid-vein depressed above and protruding below.

Internodes.—0.5 to 1.50 cm in length.

Foliar fragrance.—Subtle resinous scent of lavender oil.

Petiole.—Sessile.

Length.—Approximately 2 to 6 cm in length.

Width.—3 to 9 mm in width.

Flower:

Type of inflorescent.—Terminal spike.

Size of inflorescence.—Short and compact 1 to 2 cm in width by 2.5 to 5.0 cm in length.

Shape of inflorescence.—Broad-conical.

Peduncles.—13-23 cm in height and 1 to 2 mm in width. Unbranched with an occasional base whorl 1 to 2 cm below the main spike.

Peduncle color.—191A.

Flower shape.—Tubular.

Corolla.—Two-lipped with upper lip 2-lobed and lower lip 3-lobed.

Corolla color.—92A to 93C.

Color of flower when opening.—91A.

Calyx color.—94B with areas that are 201D to 202D.

Calyx surface.—Pubescent.

Bracteoles.—Narrow, pointed branched and 3 mm in length.

Bud color.—91B.

Fragrance.—Sweet.

Blooming period.—July and August.

Reproductive organs: Not observed.

Seed Production: Not observed.

1. A new and distinct variety of Lavendula plant named ‘Walvera’ substantially as herein illustrated and described.
